(19) United States
(12) Patent Application Publication
(10) Pub. No.: US 2021/0052825 A1
(43) Pub. Date: Feb. 25, 2021

(54) PEN NEEDLE COVER

(71) Applicant: Becton, Dickinson and Company, Franklin Lakes, NJ (US)

(72) Inventors: Cormac LYNCH, Galway (IE); Conor MEAGHER, Dublin (IE); Noel LOUGHNANE, Dublin (IE)

(73) Assignee: Becton, Dickinson and Company, Franklin Lakes, NJ (US)

(21) Appl. No.: 16/964,780

(22) PCT Filed: Jan. 30, 2019
(86) PCT No.: PCT/US2019/015827

§ 371 Date: Jul. 24, 2020

Related U.S. Application Data
(60) Provisional application No. 62/632,298, filed on Feb. 19, 2018.

(51) Int. Cl. A61M 5/32; A61F 13/40

(52) U.S. Cl. CPC A61M 5/3202; A61M 2005/3118; A61M 35/006

(57) ABSTRACT

A cover (50) is configured to enclose a pen needle or a safety pen needle (10). The cover (50) comprises a proximal end (52) in communication with an opening (58), the pen needle or the safety pen needle (10) disposed in the opening (58), a first seal (72) disposed at the proximal end (52) of the cover (50) to seal the opening (58), a cavity (56) disposed at a distal end (54) of the cover (50), a swab (60) disposed in the cavity (56), and a second seal (70) disposed at the distal end (54) of the cover (50) to seal the swab (60) in the cavity (56). The swab (60) contains at least one of an alcohol antiseptic, a non-alcohol antiseptic, and a numbing agent.

### CROSS-REFERENCE TO RELATED APPLICATION

This application claims priority under 35 U.S.C. 119(e) to U.S. provisional application Ser. No. 62/632,298, filed on Feb. 19, 2018, which is incorporated herein by reference in its entirety.

### FIELD OF THE INVENTION

The present invention relates generally to pen needle covers for enclosing pen needles used in medication delivery pens.

### BACKGROUND OF THE INVENTION

It is a well-known practice in the medical community to clean an injection site on the skin prior to administering medication, such as insulin. Antiseptic or alcohol swabs are typically used as the skin cleaning agent prior to needle injection. Generally, to perform a needle injection with a medication delivery pen, the user must carry the pen, a set of pen needles or safety per needles, and alcohol swabs.

However, using alcohol swabs poses the following problems. It is inconvenient to carry the individually packaged alcohol swabs because they take up additional space and are purchased and carried in bulk. In addition, alcohol swabs are small in size and easy to misplace. Further, many diabetics have dexterity problems which makes it difficult to open and unfurl antiseptic swabs. Although inconveniences are present, users of medication delivery pens use antiseptic or alcohol swabs for safe needle injections.

### SUMMARY OF THE INVENTION

It is an aspect of the present invention to provide a pen needle protective cover or a safety pen needle protective cover enclosed with an antiseptic or alcohol swab. Such a protective cover can be used to clean the injection site and administer medication in one package. Also, the protective cover is easy to carry around and additional products are not necessary. Finally, securing the pen needle to the medication delivery pen while the pen needle is in the protective cover allows the user to apply the swab similar to using a marker or applicator.

The foregoing and/or other aspects of the present invention can be achieved by providing a cover configured to enclose a pen needle or a safety pen needle, the cover comprising a proximal end of the cover in communication with an opening, the pen needle or the safety pen needle disposed in the opening, a first seal disposed at the proximal end of the cover to seal the opening, a cavity disposed at a distal end of the cover, a swab disposed in the cavity, and a second seal disposed at the distal end of the cover to seal the swab in the cavity.

The foregoing and/or other aspects of the present invention can also be achieved by providing a method of applying a swab to the skin of a patient via a cover configured to enclose a pen needle or a safety pen needle, the method comprising peeling off a first seal from the cover to expose the pen needle or the safety pen needle disposed within the cover, securing a medication delivery pen to the pen needle or the safety pen needle, peeling off a second seal from the cover to expose a swab, and applying the swab to the skin of the patient by gripping and moving the medication delivery pen.

Additional and/or other aspects and advantages of the present invention will be set forth in the description that follows, or will be apparent from the description, or may be learned by practice of the invention,

### BRIEF DESCRIPTION OF THE DRAWINGS

The above aspects and features of the present invention will be more apparent from the description for the exemplary embodiments of the present invention taken with reference to the accompanying drawings, in which:

FIG. 1 illustrates an exploded perspective view of a first exemplary embodiment of a pen needle assembly;

FIG. 2 illustrates a perspective view of the pen needle of FIG. 1;

FIG. 3 illustrates a top perspective view of a protective cover for the pen needle of FIG. 1;

FIG. 4 illustrates a side view of a second exemplary embodiment of a protective cover for a pen needle;

FIG. 5 illustrates a side perspective view of a third exemplary embodiment of a medication delivery pen engaged to a safety pen needle;

FIG. 6 is an exploded perspective view of the safety pen needle of FIG. 5 and a protective cover; and

FIG. 7 illustrates a top perspective view of the protective cover for the safety pen needle of FIG. 5.

### DETAILED DESCRIPTION OF EXEMPLARY EMBODIMENTS

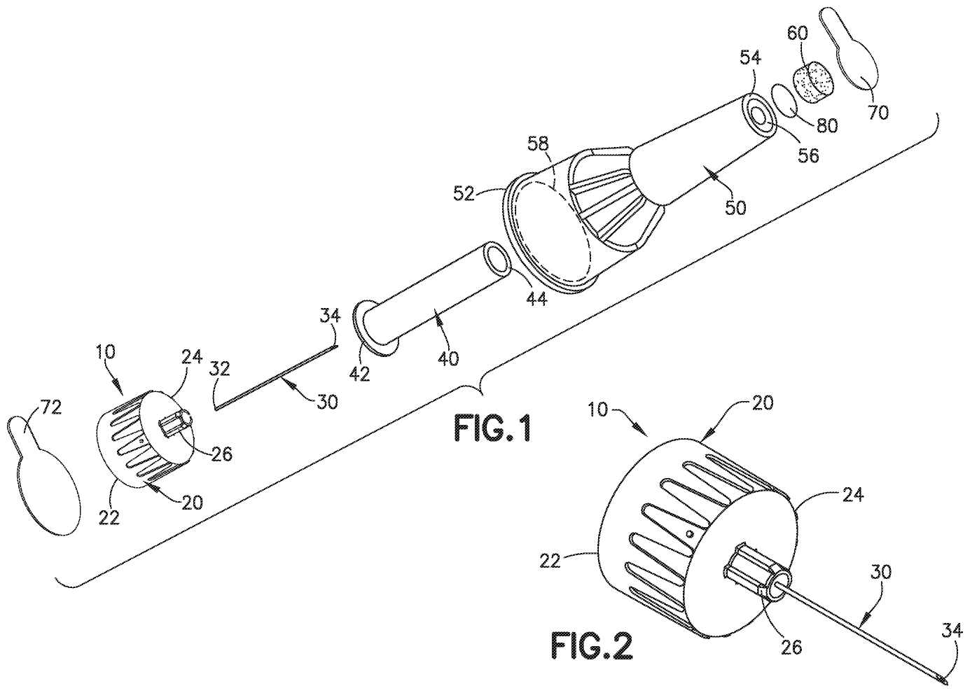

FIGS. 1-3 illustrate an exploded view of a pen needle assembly 2 comprising a pen needle 10 and a protective cover 50. As illustrated in FIGS. 1 and 2, the pen needle 10 includes a hub 20 having a proximal end 22 and a distal end 24, and a needle 30. A protrusion 26 is disposed at the distal end 24 of the hub 20 by which the needle 30 is affixed to the hub 20.

A distal end of the needle 34 is configured to be inserted into a skin of a patient and a proximal end of the needle 32 is configured to engage a medication delivery pen or an insulin pen. Exemplary pen needles 10 that can be used include the BD Ultra-Fine™ Micro 6 mm×32 G pen needle and the BD Nano™ 4 mm pen needle.

An inner shield 40 having an open proximal end 42 and a closed distal end 44 encloses the protrusion 26 and the needle 30. Further disclosure regarding pen needle 10 and the inner shield 40 and its operation can be found in U.S. Patent Publication No. 2012/0029469, which is hereby incorporated by reference in its entirety.

The pen needle assembly 2 further includes a protective cover 50 that encloses the pen needle 10 and the inner shield 40 prior to use. The material of the protective cover 50 is preferably any of a variety of plastics or polymers such as polypropylene, polyethylene or high-density polyethylene. A proximal end 52 of the protective cover 50 has an opening 58 to allow the pen needle 10 to enter. FIGS. 1, 3, 4, 6 and 7 illustrate various shapes and configurations of the protective cover 50, 150.

The opening 58 is sealed by a first seal in the form of a peelable label 72 at the proximal end 52 of the protective cover 50. The label 72 seals the pen needle 10 to maintain its sterility for transportation and security purposes prior to use with the medication delivery pen. When the pen needle 10 is ready for use, the user peels off the label 72 and removes the pen needle 10 from the protective cover 50.

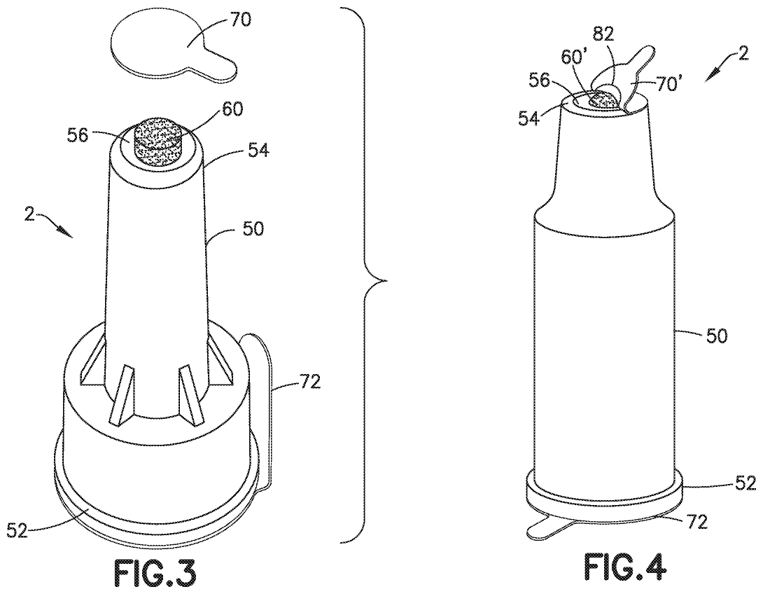

A distal end 54 of the protective cover 50 is closed to form a sealed enclosure for the pen needle 10 in conjunction with the label 72. However, FIG. 3 illustrates that on the outside, external surface of the distal end 54 of the protective cover 50, a cavity 56 is provided. The cavity 56 is not in communication with the sealed interior of the protective cover 50.

In accordance with an aspect of the present invention, the cavity 56 carries a swab 60 that is removed and used to clean the skin of the patient prior to needle insertion. The swab 60 is disposed externally to the protective cover 50 but is carried in and adhered to the cavity 56 of the protective cover 50. The swab 60 in the protective cover 50 advantageously allows the user to clean the injection site and administer medicament in one package. Also, such a configuration advantageously makes it unnecessary to purchase and carry separate swabs to clean an injection site. Accordingly, such a configuration is more convenient and advantageously takes up less space for the user to carry around.

The cavity 56 preferably includes a layer of foil 80 upon which the swab 60 rests. Specifically, the layer of foil 80 is disposed in the cavity 56 and between the swab 60 and the cavity wall. The layer of foil 80 prevents the swab 60 from drying out and forms a barrier to prevent the material of the swab 60 from seeping into the protective cover 50. This is particularly important when the swab 60 contains alcohol, since the plastic of the cover 50 can absorb alcohol over time.

The swab 60 is a porous body and acts as a sponge when compressed and released. The swab 60 is impregnated with at least one of an alcohol antiseptic, a non-alcohol antiseptic, a numbing agent, or the like. The numbing agent is particularly useful when administering medicament to children. After the injection site is sterilized, the numbing agent is applied to numb the injection site so that the needle injection process is less painful and less frightening to children. The swab is affixed to the layer of foil 80 in the cavity 56 of the protective cover 50 by at least one of bonding, ultrasonic bonding, glue that does not mix with the swab 60, heat sealing, or the like. The layer of foil 80 is affixed to the cavity wall in a similar manner.

The swab 60 is enclosed in the cavity 56 and sealed by a second seal 70 in the form of a peelable label. The label 70 preferably compresses the swab 60 in the closed position, thus making the label 70 substantially parallel to a bottom surface of the cavity 56 or to the label 72. When the label 70 is removed, the swab 60 expands and extends beyond the distal end 54 of the protective cover 50 so that it can be brought into contact with the skin of the user. The label 70 is similar to the label 72 described above. However, the labels 70, 72 separately enclose the opening 58 and the cavity 56, respectively. The labels 70, 72 are also disposed at opposite ends of the protective cover 50.

The distal end 54 of the protective cover 50 may be approximately between 4 mm and 6 mm in diameter. Ideally, a larger surface area is desired when the swab 60 is applied to the skin of the patient. A larger surface area makes it easier to spread the swab 60 at the injection site.

In another embodiment, the swab 60 is similarly affixed to the inner shield 40 and enclosed in the protective cover 50. However, the distal end 44 of the inner shield 40 may be approximately between 2 mm and 4 mm in diameter. Thus, a smaller surface area is available on the inner shield 40 compared to the distal end 54 of the protective shield 50 as described above.

The label 72 seals the pen needle assembly 2 of FIG. 3 for sale and distribution. When the pen needle assembly 2 is in used, the user removes the label 72 to expose the opening 58 of the protective cover 50 that carries the pen needle 10. Next, the user secures the pen needle 10 to the medication delivery pen using the protective cover 50.

After the pen needle 10 is engaged to the medication delivery pen, the label 70 is removed to expose the swab 60. The swab 60 automatically expands to extend beyond the distal end 54 of the protective cover 50. The user can then conveniently apply the swab 60 to the skin of the patient while gripping and moving the medication delivery pen. In this manner, the medication delivery pen advantageously acts as a handle or applicator for the swab 60. Next, the protective cover 50 is removed from the pen needle 10 and the medication delivery pen is used to perform an injection. Afterward, the pen needle 10 is removed from the medication delivery pen using the protective cover 50 and both the pen needle 10 and the protective cover 50 are discarded.

Alternately, the user can use the pen needle assembly 2 as a swab before attaching it to the medication delivery pen, by initially removing the label 70 and applying the swab 60 to the skin. The user holds the protective cover 50 to apply the swab 60 to the injection site. Subsequently, the user removes the label 72, engages the pen needle 10 to the medication delivery pen, removes the protective cover 50, and performs the injection. The protective cover 50 can then be used to remove the pen needle 10 from the medication delivery pen and both the pen needle 10 and the protective cover 50 can be discarded.

FIG. 4 illustrates a second embodiment of a protective cover 50 for a pen needle 10. This embodiment is similar to the first embodiment described above with the following modifications. FIG. 4 illustrates the material of the swab 60′ initially extending vertically out of the cavity 56 when the label 70′ is in place. The label 70′ may be provided with a shallow cavity or a recessed area 82 to accommodate this extension. Such a configuration facilitates using the pen needle assembly 2 as an applicator when preparing the skin surface of the patient for needle insertion, In other words, it is easier to apply the swab 60′ to the skin surface of the patient when the swab 60′ is large enough so that it initially extends beyond the distal end 54 of the protective cover 50. As in the previous embodiment, the label 70 includes a foil layer (not shown) to prevent absorption from the swab 60.

FIGS. 5-7 illustrate a third embodiment in which a medication delivery pen 115 is engaged to a safety pen needle 110. This embodiment is similar to the embodiments described above but involves a safety pen needle 110 instead of a conventional pen needle. Specifically, as illustrated in FIG. 5, a safety pen needle 110 is engaged to a medication delivery pen 115. Among other features, the safety pen needle 110 includes an inner shield 140 and an outer shield 145 that cover a distal end 134 of a needle 130.

When the safety pen needle 110 contacts the skin surface of the patient, the outer shield 145 retracts to expose and insert the needle 130. After use, the safety pen needle 110 is removed from the patient, the outer shield 145 extends to its original position, the inner shield 140 is activated to extend beyond the position of the outer shield 145, and the inner shield 140 is locked in place to shield the needle 130 and prevent future use of the safety pen needle 110. Further description regarding the safety pen needle 110 and its operation are provided in U.S. Pat. Nos. 8,632,503 and 9,162,030, which are hereby incorporated by reference in their entirety.

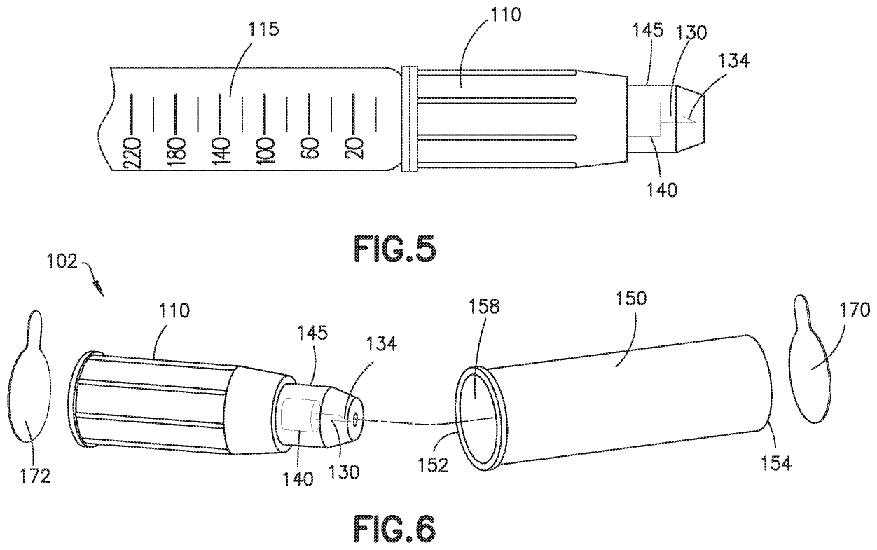

FIG. 6 illustrates a safety pen needle assembly 102 in an exploded view. The components of the safety pen needle assembly 102, such as the safety pen needle 110, the protective cover 150, and the labels 170, 172, are similarly described in the embodiments above with the following differences.

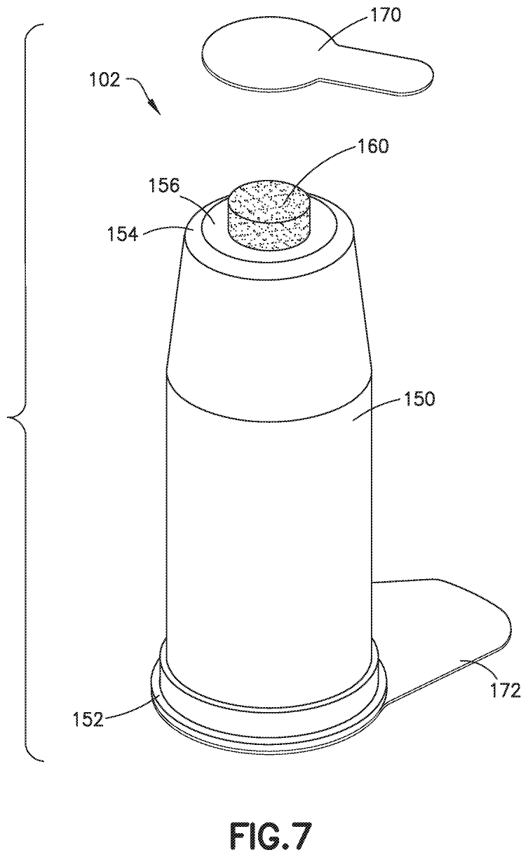

As illustrated in FIG. 7, since the safety pen needle 110 is larger than the conventional pen needle 10 of the previous embodiments, the distal end 154 of the protective cover 150 is accordingly larger. Specifically, the distal end 154 of the protective cover 150 may be approximately between 10 mm and 12 mm in diameter. In this manner, a larger surface area is advantageously present for applying the swab 160 to the skin surface of the patient.

The foregoing detailed description of the certain exemplary embodiments has been provided for the purpose of explaining the principles of the invention and its practical application, thereby enabling others skilled in the art to understand the invention for various embodiments and with various modifications as are suited to the particular use contemplated. This description is not necessarily intended to be exhaustive or to limit the invention to the precise embodiments disclosed. Any of the embodiments and/or elements disclosed herein may be combined with one another to form various additional embodiments not specifically disclosed, as long as they do not contradict each other. Accordingly, additional embodiments are possible and are intended to be encompassed within this specification and the scope of the invention. The specification describes specific examples to accomplish a more general goal that may be accomplished in another way.

As used in this application, the terms “front,” “rear,” “upper,” “lower,” “upwardly,” “downwardly,” and other orientational descriptors are intended to facilitate the description of the exemplary embodiments of the present invention, and are not intended to limit the structure of the exemplary embodiments of the present invention to any particular position or orientation. Terms of degree, such as “substantially” or “approximately” are understood by those of ordinary skill to refer to reasonable ranges outside of the given value, for example, general tolerances associated with manufacturing, assembly, and use of the described embodiments.

1. A cover configured to enclose a pen needle or a safety pen needle, the cover comprising:
a proximal end in communication with an opening, the pen needle or the safety pen needle disposed in the opening;
a first seal disposed at the proximal end of the cover to seal the opening;
a cavity disposed at a distal end of the cover;
a swab disposed in the cavity; and
a second seal disposed at the distal end of the cover to seal the swab in the cavity.

2. The cover of claim 1, wherein
the pen needle includes a needle fixed to a huh and an inner shield.

3. The cover of claim 2, wherein
the swab is disposed at a distal end of the inner shield.

4. The cover of claim 1, wherein
the safety pen needle includes an inner shield and an outer shield; and
after use, the inner shield locks to prevent reuse of the safety pen needle.

5. The cover of claim 1, wherein
the opening is not in communication with the cavity.

6. The cover of claim 1, wherein
the first and second seals comprise first and second labels, respectively.

7. The cover of claim 1, wherein
the cavity is external to the cover.

8. The cover of claim 1, wherein
the first and second seals are disposed at opposing ends of the cover.

9. The cover of claim 1, wherein
the cavity includes a foil layer to provide a liquid barrier and to prevent absorption,

10. The cover of claim 1, wherein
the swab includes at least one of an alcohol antiseptic, a non-alcohol antiseptic, and a numbing agent.

11. The cover of claim 1, wherein
the swab is fixed to the cavity by at least one of bonding, ultrasonic bonding, heat sealing and glue that does not mix with the swab.

12. The cover of claim 1, wherein
the swab is disposed between the cavity and the second seal.

13. The cover of claim 1, wherein
the swab is disposed externally to the cover.

14. The cover of claim 1, wherein
the distal end of the cover is between 4 mm and 6 mm in diameter.

15. The cover of claim 1, wherein
the distal end of the cover is between 10 mm and 12 mm in diameter.

16. A method of applying a swab to a skin of a patient via a cover configured to enclose a pen needle or a safety pen needle, the method comprising:
peeling off a first seal from the cover to expose the pen needle or the safety pen needle disposed within the cover;
securing a medication delivery pen to the pen needle or the safety pen needle;
peeling off a second seal from the cover to expose a swab; and
applying the swab to the skin of the patient by gripping and moving the medication delivery pen.

17. The method of claim 16, further comprising:
removing the cover from the pen needle or the safety pen needle after application of the swab to prepare for medication delivery.
